(19) United States
(12) Patent Application Publication
(10) Pub. No.: US 2016/0033497 A1
(43) Pub. Date: Feb. 4, 2016

(54) Luminescence methods and reagents for analyte detection

(71) Applicants: Tianxin Wang, Burlingame, CA (US); Xingxiang Li, Cary, NC (US)

(72) Inventors: Tianxin Wang, Burlingame, CA (US); Xingxiang Li, Cary, NC (US)

(21) Appl. No.: 14/816,063

(22) Filed: Aug. 3, 2015

Related U.S. Application Data
(63) Continuation-in-part of application No. 14/803,600, filed on Jul. 20, 2015.
(60) Provisional application No. 62/032,558, filed on Aug. 2, 2014.

(51) Int. Cl. G01N 33/543

(52) U.S. Cl. CPC G01N 33/54393; G01N 2333/59

(57) ABSTRACT

The present invention relates to chemiluminescent method and regent to detect analyte. One aspect of the current invention relates to using first analyte binding molecule labeled solid phase support encapsulated with chemiluminescent dye and chemiluminescent reaction generating molecule or enzyme coupled with second analyte binding molecules such as antibody HRP conjugate to detect specific analyte molecules. Another aspect of the current invention is that the analyte binding molecule labeled solid phase support encapsulated with chemiluminescent dye is magnetic.

### CROSS-REFERENCE TO RELATED APPLICATION

This application is a Continuation-In-Part application of U.S. application Ser. No. 14/803,600, filed on Jul. 20, 2015, which is a Continuation application of application Ser. No. 12/924,072. It also claims priority to U.S. Provisional Patent Application No. 62/032,558 filed on Aug. 2, 2014. The entire disclosure of the prior application is considered to be part of the disclosure of the instant application and is hereby incorporated by reference.

### TECHNICAL FIELD

The present invention relates to chemiluminescent methods and regents to detect analyte. One aspect of the current invention relates to using first analyte binding molecule labeled solid phase support (such as antibody coated micro particles) encapsulated with chemiluminescent dye and chemiluminescent reaction triggering/activating molecule/enzyme coupled with second analyte binding molecules such as antibody HRP conjugate to detect specific analyte molecules.

### BACKGROUND OF THE INVENTION

Chemiluminescence or chemoluminescence (herein sometimes referred to as CL) is the emission of light (luminescence) with limited emission of heat as the result of a chemical reaction. Besides enzyme-catalyzed chemiluminescence, small organic molecule based chemiluminescence (CL) assays are also widely used for analyte detection. Examples of chemiluminescent compounds include, but are not limited to, luminol, acridinium and its derivative, acridan and 1,2-dioxetane.

The United States Patent Application 20070264664 “ Nonseparation assay methods” described non-separation assay methods. The disclosed assay methods involve a compound capable of producing CL which is immobilized on a solid support surface as a member of a specific binding pair for capturing an analyte from a sample; an activator compound that activates the chemiluminescent compound conjugated to a specific binding pair member is added in excess along with the sample to the solid support. Addition of a trigger solution causes a chemiluminescent reaction at the sites where the activator conjugate has been specifically bound. The assay methods are termed non-separation assays because they do not require removal or separation of excess detection label (activator conjugate) prior to the detection step. The methods in the 20070264664 application are applicable to various types of assays including immunoassays, receptor-ligand binding assays and nucleic acid hybridization assays. The methods in 20070264664 application feature the use of an immobilized chemiluminescent compound and an activator compound conjugated to a specific binding partner for inducing a chemiluminescent reaction. Analyte-mediated co-localization of the chemiluminescent label compound and the activator conjugate causes the ensuing chemiluminescent reaction to take place only at the site of the bound analyte molecules. The presence of unbound, excess activator conjugate does not contribute to or interfere with the chemiluminescent reaction. As a result, the intensity of CL emitted is proportional to the quantity of analyte.

Assays performed according to the prior application involve four steps. In a first step a solid phase is provided in a test device for specifically capturing an analyte of interest. The solid phase is provided with an immobilized specific binding partner for an analyte to be detected. The solid phase is further provided with chemiluminescent labeling compound immobilized thereon. The chemiluminescent label may be provided in a number of different ways as described in the prior application. In each variant the chemiluminescent label is irreversibly attached to a substance or material in a way that renders it immobile. In a second step, the analyte-containing sample and the activator conjugate are introduced to the test device having the solid phase immobilized specific binding partner for the analyte and permitted to form specific binding complexes. They can be added separately in either order, or simultaneously, or can be pre-mixed and added as a combination. An optional delay time to allow binding reactions to occur can be inserted at this point. In the third step a triggers solution is added to produce the CL for detecting the analyte. Lastly the chemiluminescence is detected. Preferably either peak light intensity level or total integrated light intensity is measured. The quantity of light can be related to the amount of the analyte by constructing a calibration curve according to generally known methods. When light emission ensues rapidly after addition of trigger solution it is desirable to either mechanically time the onset of measurement to the addition by means of a suitable injector or to perform the addition with the test device already exposed to the detector.

The activator compound in the prior application forms part of an activator-specific binding partner conjugate. The conjugate serves a dual function; 1) binding specifically to the analyte in the assay through the specific binding partner portion, either directly or through an intermediary specific binding partner, and 2) activating the chemiluminescent compound through the activator portion. The activator portion of the conjugate is a compound that effects the activation of the chemiluminescent compound so that, in the presence of the trigger solution, CL is produced. Compounds capable of serving as the activator include transition metal salts and complexes and enzymes, especially transition metal-containing enzymes, most especially peroxidase enzymes. Transition metals useful in activator compounds include those of groups 3-12 of the periodic table, especially iron, copper, cobalt, zinc, manganese, and chromium. It should be noted that the activator molecules responsible for signal generation may operate within a physically confined radius and only have contact with a finite supply of chemiluminescent compound. This would seem to preclude large catalytic turnover in cases where the activator possesses that potential. The peroxidase which can enable the chemiluminescent reaction include lactoperoxidase, microperoxidase, myeloperoxidase, haloperoxidase, e.g. vanadium bromoperoxidase, horseradish peroxidase, fungal peroxidases such as lignin peroxidase and peroxidase from Arthromyces ramosus and Mn-dependent peroxidase produced in white rot fungi, and soybean peroxidase. Other peroxidase mimetic compounds which are not enzymes but possess peroxidase-like activity including iron complexes, such as gold nano particle, heme, and Mn-TPPS₄ are known which catalyze the chemiluminescent oxidation of substrates are explicitly considered to be within the scope of the meaning of peroxidase as used herein. The activator produces highly active oxygen and/or active radical which can be consumed by the nearby chemiluminescent compound to generate detectable CL signal. However, not only the activator which had already formed binding complex with the analyte and the chemiluminescent compound produce highly active oxygen and/or active radical at the presence of trigger solution; in fact, all the activator compounds including the those in the unbound form produce highly active oxygen. These highly active oxygen and active radical will also be consumed by chemiluminescent compound in the non bound form and generate background light signals or noise. These light signals are not related to the presence of analyte therefore become background (noise) signal in the assay.

### SUMMARY OF THE INVENTION

The present invention relates to chemiluminescent methods and regents to detect analyte. One aspect of the current invention relates to using chemiluminescent molecule/enzyme coupled with analyte binding molecules to detect specific analyte molecules involving encapsulated chemiluminescent (bioluminescent) dye binding pair-chemiluminescence triggering/activating molecule binding pair system.

### DETAILED DESCRIPTION OF THE INVENTIONS

The current invention provides a method to improve the performance of none separation assay described in the prior application by increase the signal/background ratio of the assay. The signal in the signal/noise (background) ratio is produced by the analyte. The definition of signal/noise (background) ratio is well known to the skilled in the art. The principle is by adding highly active oxygen/active free radical scavenger such as antioxidant/free radical scavenger (e.g., VC, VE, BHT and Cysteine) or enzymes that can decompose the highly active oxygen, e.g. superoxide dismutase (SOD, e.g. S8160 or S5389 from Sigma) to the assay mix, the background will decrease. In the bound form the highly active oxygen/radical is produced in very close proximity to the chemiluminescent compound and, consequently, has better chance to react with the chemiluminescent compound generating analyte-dependent light signal than being consumed by antioxidant/free radical scavenger/decomposing enzymes. Therefore the signal produced by analyte is less likely to be decreased. There are many antioxidant/free radical scavenger/reductant, which can be used in the assay. Examples of scavenger/reductant include, but are not limited to, glutathione, vitamin C, and vitamin E, coenzyme Q, thiol containing compounds, melatonin, thiols or polyphenols (e.g. tea polyphenol). The skilled in the art can find many of them and the optimal one or combination thereof can be found by screening them in the assay for the highest signal/noise ratio. There are several different types of active oxygen/radical such as superoxide anion and hydroxyl radical. Different CL compounds may be triggered by different type of active oxygen therefore require different antioxidant/free radical scavenger/reductant for an assay. For example, SOD may be suitable for luminol-based assay while catalase maybe suitable for the acridan-based assay.

The optimal type and amounts of scavenger/reductant in a reaction mix can be determined experimentally. In general, the assay is performed according to the protocol described in the prior application. Varying amounts of antioxidant/free radical scavenger/enzyme that can decompose the highly active oxygen are added to the assay mix (e g immediately before the adding of the trigger solution) and the amount which gives the best signal/noise ratio is selected as the optimal concentration. For example, in order to find an optimal concentration of antioxidant VC or TP (tea polyphenol) or SOD, the assay can be carried out in reaction mixes containing a final concentration of 1 ng/ml, 10 ng/ml, 100 ng/ml, 1 microgram/ml, 10 micrograms/ml, 100 micrograms/ml, 1 mg/ml, or 10mg/ml. The concentration, which gives the best signal/noise ratio, is selected. The optimal concentration can be further refined using smaller incremental concentrations. For example, if 10 microgram/ml gives the best result in the first experiment, concentrations of 2.5 microgram /ml, 5 microgram/ml, 10 microgram/ml, 15 microgram/ml and 20 micrograms/ml can be further tested to see if better result can be obtained at more refined concentrations, e.g., 12.5 micrograms/ml.

Use of a scavenger in the reaction allows the chemiluminescent compound to be conjugated to a specific binding partner directly instead of being immobilized on a solid support. In an example an assay where acridan, which is the chemiluminescent compound, is directly coupled to a first binding partner (e.g., an n antibody) and the resulting conjugate used in the reaction without immobilizing acridan and antibody on a solid support such as an Amberlite microparticle. In this example, the analyte activator (e.g. enzyme HRP) is conjugated to a second binding partner (e.g., the second antibody). In the presence of an analyte, which can bind to both first and second binding partners, the chemiluminescent compound acridan and HRP forms a complex via binding between the analyte and binding partners. Upon addition of a trigger solution, the acridan in the complex is activated. This acridan directly coupled with antibody is not immobilized. The complex formed would be chemiluminescent compound conjugated to a specific binding pair member—analyte-activator compound conjugated to another specific binding pair member instead of immobilized chemiluminescent compound conjugated to a specific binding pair member—analyte-activator compound conjugated to another specific binding pair member. Therefore, no solid phase support is required.

In one embodiment of microparticle immunoassay using labeled amberlite microparticles, the procedure and reagents are identical to the Example 15 of 20070264664 application except the adding of antioxidant/free radical scavenger. Take 100 microgram/reaction of acridan and antibody co-labeled Amberlite microparticles of example 10 from 20070264664 application. The particles are washed three times with 1× PBS+0.05% Tween-20 and resuspended in sheep anti-mouse IgG F(ab¹)₂-HRP conjugate diluted 1:1.2×10⁶ in 1× PBS buffer containing 1% BSA and 1% sucrose. The particle suspension is dispensed into 26 wells of a white polystyrene 96 well plate. IgG standards in sheep anti-mouse IgG F(ab¹)₂ -HRP conjugate solution are prepared by 2-fold serial dilution to result in final concentrations of 100 ng/mL-0.048 ng/mL or 0 ng/mL in the wells. The respective standards and zero are dispensed into wells to make a final reaction volume of 50 uL/well. The plate is incubated for 1 hr at room temperature on a plate shaker. Next optimal amount of antioxidant/free radical scavenger is added. The plate is transferred to a plate luminometer. Without removing the conjugate solution, luminescence is generated by sequentially injecting 100 uL of trigger solution, and reading the integrated intensity in each well for 5 seconds. A plot of the resulting assay allows quantitation of IgG. The optimal amount of antioxidant/free radical scavenger can be determined experimentally using the above procedure, for example, if VC is used, 2-fold serial dilution to result in final concentrations of 500 ng/mL-0.048 ng/mL or 0 ng/mL in the wells of VC is added to the wells containing either no analyte or 50 ng/ml analyte and the VC concentration give the best signal/noise ratio is selected as optimal amount. One suitable concentration of VC is to add 10 microliter of ascorbic acid solution (10 mM) before injection of 100 microliter of trigger solution.

In another embodiment, assay is performed in a 96-well plate by adding 20 microliter of acridan-labeled mouse anti-human IL-6 antibody (2.0 microgram/mL in 1× PBS), 20 microliter of HRP-conjugated mouse antihuman IL-6 antibody (2.0 microgram/mL in 1× PBS, the two antibodies bind to different regions of IL-6), and 30 microliter of sample solution; after 30 min incubation at room temperature; addition of 10 microliter of ascorbic acid solution (10 mM) before injection of 100 microliter of trigger solution and reading is performed for 5 seconds.

In another embodiment of microplate immunoassay using unlabeled capture antibody and labeled BSA, the procedure and reagents are similar to the Example 13 of 20070264664 application except the adding of antioxidant/free radical scavenger or SOD and luminol is used instead of acridan. A 50 uL aliquot of unlabeled sheep anti-mouse IgG (H+L) 40 microgram/mL of 1× PBS is added to coat each of 26 wells of a white polystyrene 96 well plate. The plate is agitated for 5 minutes at room temperature on an orbital shaker. The solution is removed and the wells washed three times with lx.PBS+0.05% Tween-20, removing all wash buffer after each step. The luminol labeled BSA (10%) is diluted in 50 uL/mL in PBS buffer+1% sucrose. A 100 uL aliquot is added to each of 26 wells of a white polystyrene 96 well plate. The plate is held for 1 hr at 37 degree. The solution is removed and the wells washed three times with PBS+0.05% Tween-20, removing all wash buffer after each step. Sheep anti-mouse IgG F(ab¹)₂-HRP conjugate is diluted 1:1.2.times.10⁶ in a conjugate buffer comprising 1% BSA and 1% sucrose in 1×.PBS. Aliquots of diluted conjugate and suitable amount of antioxidant/free radical scavenger are dispensed into the 26 wells. IgG standards containing from 100 ng/mL-0.048 ng/mL were prepared by 2-fold dilution along with a 0 ng/mL solution in anti-IgG F(ab¹)₂-HRP conjugate solution. The standards and zero are dispensed into wells achieving a final volume 50 uL/well. The plate is incubated 1 hr at room temperature on the plate shaker. The plate is transferred to a plate luminometer. Without removing the conjugate solution, luminescence is generated by sequentially injecting 100 uL of trigger solution (the trigger solution use 1 uM p-iodophenol instead of the p-hydroxy-cinnamic acid), and reading the integrated intensity in each well for 5 seconds. Certain p-nitrophenol derivatives such as p-nitrophenol, o-methoxyphenol, p-methoxyphenol and 4-hydroxy-3-methoxycinnamic acids can also be antioxidant/free radical scavenger for this kind of assay.

Alternatively, luminol can be used instead of acridan in the above examples.

In another embodiment, assay is performed in a 96-well plate or test tube by adding 20 microliter of 0.5 um diameter polystyrene microparticle coated with mouse anti-human IL-6 antibody (10 microgram/mL in 1× PBS, the microparticle is encapsulated with luminol or acridan or acridinium ester), 20 microliter of HRP-conjugated second mouse anti human IL-6 antibody (2.0 microgram/mL in 1× PBS, the second antibody bind with different region of IL-6), and 30 microliter of sample solution; after 10 min incubation at room temperature; addition of 10 microliter of ascorbic acid solution (10 mM) before injection of 100 microliter of trigger solution (0.1M sodium borate solution, pH 10.0 containing of 5.00×10⁻⁴ M H₂O₂ and 5.00×10⁻⁴ M para-iodophenol) and reading is performed for 50 seconds. Alternatively, the reading can be performed 1 second later after the addition of trigger solution and read for 50 seconds; delayed reading can reduce the interference of light generated from reagents mixing. Alternatively, instead of using the HRP-conjugated mouse antihuman IL-6 antibody in the assay above, 20 microliter of 50 nm or 100 nm diameter polystyrene microparticle coated with both HRP and the second mouse antihuman IL-6 antibody (2.0 microgram/mL in 1× PBS, the second antibody bind with different region of IL-6) can be used for the assay. The ratio between HRP: the second mouse antihuman IL-6 antibody can be 0.1˜10.

Preferably, the microparticle suitable for the current invention is polymer based such as varieties of micro spheres. The preferred make of microspheres is polystyrene or latex material. However, any type of polymeric make of microspheres is acceptable including but not limited to brominated polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethyhnethacrylate, or combinations thereof. The polymeric bead can be made easily by polymerization of monomers such as varieties of acrylates, styrenes, diene compounds or their derivatives. Suitable microspheres and the making of them are also available from U.S. Pat. Nos. 6,649,414; 6,514,295; 5,073,498; 5,194,300; 5,356,713; 4,259,313; 4,283,382 and the reference cited in these patents. Many vendors (such as Cortex biochem Inc, CA; Seradyn, Inc. IN; Dynal Biotech Inc., NY; Spherotech, Inc. IL; Bangs Laboratories, Inc. IN; Polysciences, Inc. PA) also provide suitable microspheres and micro particles and provide customer manufacture service. The microspheres can be either non cross linked or cross linked (such as contain 0.1 to 30% of a cross-linking agent, e.g. divinyl benzene, ethylene glycol dimethacrylate, trimethylol propane trimethacrylate, or N,N′ methylene-bis-acrylamide or other functionally equivalent agents known in the art). Preferably the microparticle is uniform in size and shape and preferably each microparticle contains the same or similar amount of signal groups/compounds for high sensitive detection of certain analyte. A purification step (e.g. centrifugation, filtration, size exclusion column) could be performed to purify non-uniform microparticles (e.g. those made from milling) into highly uniform micro particles.

When microparticles or the like are used, chemiluminescent (signaling) moieties can be encapsulated in the particles besides coated on the surface of the particles. Encapsulation may be performed through physical means, e.g., trapping, internal adsorption, or through chemical means, e.g., covalent coupling. Alternatively, signaling moieties can first be directly or indirectly coupled to a carrier (e.g., a polymer or nanoparticles) and then encapsulated in the particle. The encapsulated signaling moieties could be in the form of aggregate, e.g., small particles, powder, or crystals, which are preferably in nano meter size range. Suitable chemicals for encapsulating chemiluminescent or fluorescent compounds include, but are not limited to, polymers such as polystyrene. Suitable encapsulation procedure can be found in U.S. Pat. Nos. 6,649,414; 6,514,295; 5,073,498; 5,194,300; 5,356,713; 4,259,313; 4,283,382 and the reference cited in these patents. Many vendors (such as Cortex biochem Inc, CA; Seradyn, Inc. IN; Dynal Biotech Inc., NY; Spherotech, Inc. IL; Bangs Laboratories, Inc. IN; Polysciences, Inc. PA) also provide encapsulated microspheres and encapsulation service. Signaling moieties (molecule) can be derivatized (such as attaching a lipophilic group to it) for high encapsulation rate.

Alternatively, chemiluminescent compound (or their derivatives) having reactive groups (such as amine group or carboxyl group) may be coupled to monomers containing reactive group (such as 4-amino styrene) and then polymerized or copolymerized to give encapsulated micro sphere. Alternatively, the microsphere can be made to have reactive group (such as amine group) inside (such as those generated from 4-amino styrene) and then couple chemiluminescent or fluorescent compound (or their derivatives, such as acridinium NHS ester or acridan NHS ester) with reactive groups (such as carboxyl group) to the micro sphere. The resulting microspheres will have chemiluminescent or fluorescent compound covalently encapsulated inside. The surface of the resulting microspheres can be modified for with analyte binding groups coupling.

Encapsulation of acridinium, luminol or acridan into microparticle: This example teaches a method for encapsulating acridinium or luminol or acridan, or their derivatives, into microparticle that can be used to coat with affinity molecule for the assay. A sufficiently hydrophobic moiety or moieties can be attached to acridinium, luminol or acridan, thereby creating an acridinium, luminol or acridan derivative that is sufficiently hydrophobic, i.e., low solubility in aqueous solution. Such a hydrophobic acridinium or luminol or acridan species minimizes the leaching from inside the microparticles under aqueous conditions once it is encapsulated inside the microparticles.

Hydrophobic acridinium or luminol or acridan derivatives are normally dissolved in an organic solvent so that a concentrated acridinium or luminol or acridan solution can be created for encapsulation. The microparticles used for encapsulation should be compatible with the organic solvent, i.e., the basic structure of microparticles should not be partially or completely dissolved, or otherwise significantly altered. For example, polystyrene-based microparticles may be dissolved in certain organic solvents such as chloroform, but would be compatible with the others such as ethanol. Polystyrene microparticles copolymerized with a cross-linking polymer(s) may be compatible with most organic solvents, including dichloromethane and chloroform. The microparticles are preferably, but not necessarily, functionalized with functional groups such as primary amine or carboxyl group. The following procedure teaches one method for encapsulating acridinium or its derivatives in microparticles. It is understood that different methods, or variations of the current method, can also be used to achieve sufficient encapsulation of acridinium or luminol or acridan or its derivatives.

This procedure teaches the encapsulation of an acridinium derivative, 4-dodecylphenyl-10-methylacridinum-9-carboxylate trifluoromethane sulfonate, in a cross-linked polystyrene microparticles (Spherotech, Inc, catalog number APX-20-10, 2.48 micrometer in diameter), which is functionalized with primary amines. Prior to encapsulation, the microparticles are washed twice with dry ethanol (100%) and then twice with acetone. One hundred milligrams of the acridinium derivative is dissolved in 1.0 mL CH2Cl2 (dichloromethane) to make 10% (100 mg/mL) solution, which is used to suspend 50 mg cross linked polystyrene particles prepared as described above. The microparticle solution is then rotated in a rotating device for 30 minutes at room temperature. The microparticles are then filtrated through a 0.1 micrometer VVPP filter (Millipore catalog number VVLP04700) and washed with 10 mL of 50% ethanol aqueous solution three times. The microparticles are then dialyzed in 1 LPBS buffer ovenight to remove unencapsulated acridinium. The dialysis step is repeated once or more. The resulting microparticles are now encapsulated with the acridinium. The functional groups on the microparticles surface can be used to directly or indirectly couple with analyte binding moieties. The resultant microparticles can be used for analyte detection. Alternatively, luminol or acridan or their alkyl derivative instead of acridinium can be used in the same way to be encapsulated in the microparticles.

In another example, Polystyrene latex particles (175 nm in diameter) having about 8.3 carboxyl groups per nm2 of surface (Bangs Laboratories, Carmel, Ind.) is stirred with 3.3 mM acridan in 8:1:1 (vol/vol) ethylene glycol/benzyl alcohol/water for 8-10 min at 110° C. The particles are diluted with equal volumes of ethanol, centrifuged, washed with water, and sonicated. A similar procedure is used to incorporate dioxetane dye. Next maleimide substituted dextran is prepared from amino-dextran and sulfo-SMCC followed by dialysis. Dyed 175-nm latex particles are coupled to the amino-dextran with EDAC followed by centrifugation, washing, and sonication. After incubation with 1 mg/ml thiolated antibody for 16 h at 20° C., the reactive groups are capped with mercaptoacetic acid followed by excess iodoacetic acid. And then the beads are washed with 1× PBS 3 times.

The solid phase used for encapsulation of dye can also be the wall of the plastic test tube or microwell plate. In one example, polystyrene microwell plate for ELISA is used. The dye (either acridan or acridinium or luminol or their derivative) is dissolved in ethanol to make a saturated solution. Next the solution is added to the well and sealed and incubated at 50 min for 2 h. Optionally the plate is sonicated. The plate is washed with 0.1% Triton-100 in 1× PBS 3 times, 1× PBS 3 times and then coated with antibody and then blocked with BSA. The coating of antibody and blocking is performed as the same as regular ELISA. The plate now is ready for the assay. To perform the assay, 20 microliter of HRP-conjugated antibody (2.0 microgram/mL in 1× PBS, this antibody bind to a different region of the target as the coating antibody), 50 ul 0.1% BSA in 1× PBS and 30 microliter of sample solution; after lhr incubation at room temperature; addition of 10 microliter of ascorbic acid solution (1 mM) before injection of 100 microliter of trigger solution and reading is performed for 5 seconds. If the detector is facing the bottle of the plate, colored dye that can absorb the chemiluminescent light can be added to reduce the background.

Optionally, the activator (e.g. HRP) and the affinity group can also be coated on the surface of microparticle and be used in the assay in the current invention instead of using the activator-affinity group conjugate. Preferred microparticle size is between 50 nm-500 nm.

The chemiluminescent dye particle can also be magnetic particle. If magnetic particle is used, after the incubation and before the addition of triggering solution, magnetic field can be applied to move the magnetic particle to one side of the wall of the test tube or well. The detector will face the wall having the magnetic particle attached. This can reduce the back ground of the assay. Colored dye that can absorb the chemiluminescent light can be added to reduce the background.

The affinity group or groups can be any chemical or biological functionality with affinity for certain analytes. They include, but are not limited to, DNA, PNA (peptide nucleic acid), polynucleotides, antibody, antigen, aptamers, chelator, metals, lipophilic molecules, hydrophilic molecules, ionic molecules (such as acidic and basic molecules), dendrimer, polymers having affinity groups and other structures having specific affinity interactions with certain analytes. Preferably affinity group(s) is selected from antibody and aptamers. They can also be nucleic acid if the target analyte is nucleic acid. In addition to antibody-based and nucleic acid-based systems, other specific binding pairs as are generally known to one of ordinary skill in the art of binding assays can serve as the basis for test methods according to the present invention. The fluorescein/anti-fluorescein, digoxigenin/anti-digoxigenin, and nitrophenyl/anti-nitrophenyl pairs are exemplary.

The chemiluminescent dye binding pair also has chemiluminescent moiety that can generate chemiluminescent light under suitable condition. The chemiluminescent moiety can be chemiluminescent group(s) or chemiluminescent molecule(s) or chemiluminescent particle. The chemiluminescent binding pair can be in the form of solid phase; e.g, both chemiluminescent moiety and affinity group(s) are immobilized on solid support surface or the affinity groups are immobilized on solid support surface while the chemiluminescent moiety is encapsulated inside the solid support such as microparticle. The chemiluminescent binding pair can also be chemiluminescent moiety and affinity group(s) directly coupled together or conjugated together though a linker.

Chemiluminescent moiety may be coupled to the carrier either permanently (non-releasable) or through a cleavable (releasable) bond, e.g., photo-labile bond, chemical-labile bond such as an acid sensitive bond or a detachable bond, e.g., polynucleotide base pairing. The affinity group may be indirectly coupled to the carrier through a linker or an adaptor through, for example, a ligand-receptor binding (e.g., biotin-avidin) or hybridization between a polynucleotide and its complementary sequence. The carrier entity can be a polymer, a microparticle, or a combination of the two. Appropriate natural or synthetic polymers include, but are not limited to, oligomers (such as peptides), linear or cross-linked polymers (such as poly lysine, poly acrylic acid, proteins) or highly branched macromolecules (such as dendrimers). A chemical, biological or physical entity can be used as a carrier as long as it has multiple functional groups that allow direct or indirect conjugation of multiple numbers of signal (chemiluminescent/fluorescent) compounds/groups and affinity groups. The more functional groups a carrier has, the better amplification it will provide. One of the preferred carriers is a microparticle because it can be coated with a large number of functional groups such as carboxyl group or primary amine. Preferred size of microparticles is in the range of nanometer to micrometer in diameter. Suitable microparticles include, but are not limited to, microspheres, nanoparticles, liposomes, microcapsules and etc. In some embodiments, preferred size of particle is between 5 nanometers (nm) to 100 micrometers (μm) in diameter. Many vendors (e.g. Bangslabs Inc, Spherotech, Inc. Seradyn, Inc.) provide particles suitable for current invention.

Solid supports useful in the practice of the present invention can be of various materials, shapes, and sizes. Materials already in use in binding assays including microwell plates of the 96-well, 384-well or higher number varieties, test tubes, sample cups, plastic spheres, cellulose, paper or plastic test strips, latex particles, polymer particles, silica particles, magnetic particles, especially those having average diameters of 5 nm-100 um, and nanoparticles of various materials can all provide a useful solid support for attachment of chemiluminescent groups and for immobilizing specific affinity groups. Preferably, the microparticle is polymer based such as varieties of micro spheres. The preferred make of microspheres is polystyrene or latex material. However, any type of polymeric make of microspheres is acceptable including but not limited to brominated polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethyhnethacrylate, or combinations thereof. The polymeric bead can be made easily by polymerization of monomers such as varieties of acrylates, styrenes, diene compounds or their derivatives. Suitable microspheres and the making of them are also available from many patents such as U.S. Pat. No. 6,649,414 and the reference they cited. Many vendors (such as Cortex biochem Inc, CA; Seradyn, Inc. IN; Dynal Biotech Inc., NY; Spherotech, Inc. IL; Bangs Laboratories, Inc. IN; Polysciences, Inc. PA) also provide suitable microspheres and micro particles and provide customer manufacture service. The microspheres can be either non cross linked or cross linked (such as contain 0.1 to 30% of a cross-linking agent, e.g. divinyl benzene, ethylene glycol dimethacrylate, trimethylol propane trimethacrylate, or N,N′ methylene-bis-acrylamide or other functionally equivalent agents known in the art). Preferably the microparticle is uniform in size and shape and preferably each microparticle contains the same or similar amount of signal groups/compounds for high sensitive detection of certain analyte. The microparticle may have additional surface functional groups that include, but are not limited to, carboxylates, esters, alcohols, carbamides, aldehydes, amines, sulfur oxides, nitrogen oxides, or halides. The functionality of the microparticles' surface groups gives the microparticle their coupling capability allowing chemical binding of analytical reactants. In addition to functional groups on microparticles the signal molecules such as dyes themselves can also carry chemically reactive functional groups, which in addition to groups listed above, can also be carboxylic acid, carboxylic acid succinimidyl ester, carboxylic acid anhydride, sulfonyl chloride, sulfonyl fluoride, hydrazine derivatives, acyl azide, isocyanate, haloacetamide, phenols, thiols, and ketones. These functional groups are useful for attachment of analytical reactants, i.e., classical, commonly used reactants such as antibody, antigen (hapten), digoxigenin, or nucleic acid probe. These may also include reactants that can form specific, high-affinity conjugates such as avidin-biotin, receptor-ligand, ligand-ligate, enzyme-substrate, lectin-carbohydrate, protein A-immunoglobulin, etc.

A carrier may be first directly or indirectly coupled with signal group (such as chemiluminescent compounds or fluorescent compounds) or affinity groups or both and then directly or indirectly conjugated to yet another carrier of the same type or different type. For example, acridinium and an oligonucleotide probe can be first coupled to polylysine to generate an acridinium-polylysine-oligonucleotide probe complex, which is subsequently conjugated to a microparticle. This carrier-to-carrier coupling reaction can be repeated a number of times to achieve further amplification prior to, during, or after contacting the analyte. When microparticles or the like are used as carriers, signal groups such as chemiluminescent compounds may be encapsulated in the particles. Encapsulation may be performed through physical means, e.g., trapping, internal adsorption, or through chemical means, e.g., covalent coupling. Alternatively, signal groups such as chemiluminescent compounds can first be directly or indirectly coupled to a carrier (e.g., a polymer or nanoparticles) and then encapsulated in the particle. The chemiluminescent compounds encapsulated could be in the form of aggregate, e.g., small particles, powder, or crystals, which are preferably in nanometer size range. For example, when rare-earth element such as Eu is used, it could be in the form of Eu metal particles, Eu oxide particles or other Eu containing compounds aggregate. Suitable chemicals for encapsulating chemiluminescent or fluorescent compounds include, but are not limited to, polymers such as polystyrene and small organic compounds such as Si containing compounds can also be used to coat chemiluminescent or fluorescent compound particle to give the encapsulated chemiluminescent microparticles. Many vendors (such as Cortex biochem Inc, CA; Seradyn, Inc. IN; Dynal Biotech Inc., NY; Spherotech, Inc. IL; mBangs Laboratories, Inc. IN; Polysciences, Inc. PA) also provide encapsulated microspheres and encapsulation service. It is normally, but not always, preferred that large numbers of signal molecules, or their derivatives, are encapsulated in microparticles to increase the sensitivity. Signal molecule can be derivatized (such as attaching a lipophilic group to it) for high encapsulation rate.

Alternatively chemiluminescent compound (or their derivatives) having reactive groups (such as amine group or carboxyl group) can also be coupled to monomers containing reactive group (such as 4-amino styrene) and then polymerized or copolymerized to give encapsulated micro sphere. Alternatively, the microsphere can be made to have reactive group (such as amine group) inside (such as those generated from 4-amino styrene) and then couple chemiluminescent or fluorescent compound (or their derivatives, such as acridinium NHS ester) with reactive groups (such as carboxyl group) to the micro sphere. The resulting microspheres will have chemiluminescent or fluorescent compound covalently encapsulated inside. The surface of the resulting microspheres can be modified for with affinity groups coupling. Cross-linked microspheres are more convenient for encapsulation and internal coupling since they normally cannot be dissolved in organic solvent. If non-cross linked micro sphere is used, the solvents during process need to be carefully examined to avoid dissolution. More examples of structure that can be used as chemiluminescent or fluorescent binding pairs can be found in US patent application 20050191687.

Appropriate CL by the chemiluminescent moiety here includes, but is not limited to, both direct chemiluminescence such as that generated with enzymes or acridinium and electro chemiluminescence such as that generated with rare earth elements. Chemiluminescent molecule(s)/compound(s) can be anything that generates light signal under appropriate conditions or the precursors that gives rise to such compounds. Examples for this type of compounds include both chemiluminescent compound (e.g., acridinium, proteins that can generate light, enzymes that can catalyze CL reaction) and electrochemolumincescent agents (e.g., certain organic compounds or metal elements in appropriate chelators).

In some embodiments the preferred chemiluminescent compounds are capable of being oxidized to produce CL in the presence of the activator and/or a trigger solution. An exemplary class of compounds which by incorporation of a linker and reactive group could serve as the chemiluminescent label include luminol, and structurally related cyclic hydrazides including isoluminol, aminobutylethylisoluminol (ABEI), aminohexylethylisoluminol (AHEI), 7-dimethylaminonaphthalene-1,2-dicarboxylic acid hydrazide, ring-substituted aminophthalhydrazides, anthracene-2,3-dicarboxylic acid hydrazides, phenathrene-1,2-dicarboxylic acid hydrazides, pyrenedicarboxylic acid hydrazides, 5-hydroxy-phthalhydrazide, 6-hydroxyphthalhydrazide, as well as other phthalazinedione analogs disclosed in U.S. Pat. No. 5,420,275 and in U.S. Pat. No. 5,324,835. Another class of chemiluminescent moieties include acridinium/acridan esters, thioesters and sulfonamides disclosed in U.S. Pat. Nos. 5,491,072 and 6,030,803. Another class of chemiluminescent moieties includes the heterocyclic compounds disclosed in U.S. Pat. Nos. 5,922,558 and 6,891,057 as well as US patent application 20070264664. It is considered that any compound known to produce CL by the action of hydrogen peroxide and a peroxidase will function as the chemiluminescent moiety used in the present invention. Numerous such compounds of various structural classes, including xanthene dyes, aromatic amines and heterocyclic amines are known in the art to produce CL under these conditions. Other examples of chemiluminescent compounds (e.g. dioxetane type molecules, luciferin) can be found from well-known patent, literatures and commercial venders. For example, lumigen provides many chemiluminescent compounds (e.g. Lumigen PS-atto, Lumigen PS-2, Lumigen PS-3, Lumigen TMA-6, Lumigen TMA-3 can be used to emit light when mixed with peroxidase).

The activator is a compound/particle/solid phase that effects the activation of the chemiluminescent compound so that, in the presence of the trigger solution (sometimes no trigger is required), CL is produced. Compounds capable of serving as the activator include transition metal salts and complexes and enzymes, especially transition metal-containing enzymes, most especially peroxidase enzymes. Transition metals useful in activator compounds include those of groups 3-12 of the periodic table, especially iron, copper, cobalt, zinc, manganese, and chromium. Gold nanoparticles can also be used as activators. It should be noted that the activator molecules responsible for signal generation may sometimes operate within a physically confined radius and only have contact with a finite supply of chemiluminescent compound. This would seem to preclude large catalytic turnover in cases where the activator possesses that potential. The peroxidase which can undergo the chemiluminescent reaction include lactoperoxidase, microperoxidase, myeloperoxidase, haloperoxidase, e.g. vanadium bromoperoxidase, horseradish peroxidase, fungal peroxidases such as lignin peroxidase and peroxidase from Arthromyces ramosus and Mn-dependent peroxidase produced in white rot fungi, and soybean peroxidase. Other peroxidase mimetic compounds which are not enzymes but possess peroxidase-like activity including iron complexes, such as heme, and Mn-TPPS₄ are known which catalyze the chemiluminescent oxidation of substrates are explicitly considered to be within the scope of the meaning of peroxidase as used herein. Conjugates or complexes of a peroxidase and a biological molecule can also be used in the method for producing CL, the only proviso being that the conjugate display peroxidase activity. Biological molecules which can be conjugated to one or more molecules of a peroxidase include DNA, RNA, oligonucleotides, antibodies, antibody fragments, antibody-DNA chimeras, antigens, haptens, proteins, lectins, avidin, streptavidin and biotin. Complexes including or incorporating a peroxidase, such as liposomes, micelles, vesicles and polymers which are functionalized for attachment to biological molecules, can also be used in the methods of the present invention. Other example of activator can be found in US patent application 20070264664. The trigger solution provides a reactant necessary for generating the excited state compound necessary for CL. The reactant may be one necessary for performing the chemiluminescent reaction by reacting directly with the chemiluminescent label. It may serve instead of or in addition to this function to facilitate the action of the activator compound. This will be the case, for example, when the activator is a peroxidase enzyme. In a preferred embodiment the trigger solution comprises a peroxide compound. The peroxide component is any peroxide or alkyl hydroperoxide capable of reacting with the peroxidase. Preferred peroxides include hydrogen peroxide, urea peroxide, and perborate salts. Incorporation of certain enhancer compounds into the trigger solution promotes the CL of the enzyme. Included among these enhancers are phenolic compounds and aromatic amines known to enhance other peroxidase reactions. Preferred enhancers include but are not limited to: p-phenylphenol, p-iodophenol, p-bromophenol, p-hydroxycinnamic acid, p-imidazolylphenol, acetaminophen, 2,4-dichlorophenol, 2-naphthol and 6-bromo-2-naphthol. Mixtures of more than one enhancer from those classes mentioned above can also be employed. The trigger solution can also contain one or more detergents or polymeric surfactants to enhance the luminescence efficiency of the light-producing reaction or improve the signal/noise ratio of the assay. Nonionic surfactants useful in the practice of the present invention include by way of example polyoxyethylenated alkylphenols, polyoxyethylenated alcohols, polyoxyethylenated ethers and polyoxyethylenated sorbitol esters. Monomeric cationic surfactants, including quaternary ammonium salt compounds such as CTAB and quaternary phosphonium salt compounds can be used. Polymeric cationic surfactants including those comprising quaternary ammonium and phosphonium salt groups can also be used for this purpose. More examples of trigger can be found in US patent application 20070264664.

In some embodiments, the chemiluminescent compound described above is used to form the chemiluminescent binding pair. Examples of preferred chemiluminescent compounds include chemiluminescent acridan, acridinium, ABEI, luminol, isoluminol and dioxetane type compounds. Examples of these chemiluminescent binding pair include these chemiluminescent compounds labeled antibody, antigen, nucleic acid or aptamer such as acridan labeled antibody, acridinium labeled antibody, luminol labeled nucleic acid and etc. They can also be in the form of chemiluminescent compounds and affinity groups immobilized on solid form as described above such as acridinium and antibody co immobilized on microsphere and acridan and antibody co immobilized on micro well plate as described in 20070264664 application. The activator and/or trigger that can initiate the CL can be in the reaction solution. For example, H₂O₂ other peroxide and NaOH for acridinium, peroxidase and H₂O₂ or other peroxide for luminol and acridan. The activator and/or trigger can be added at the beginning of the assay or right before the detection step after the binding is performed.

The above embodiments can cause acridan as chemiluminescent moiety. Other chemiluminescent compounds can also be used instead (e.g . acridinium, ABEI, luminol, isoluminol and dioxetane). For example, luminol and antibody co-labeled Amberlite microparticles can used in the above example instead of using acridan and antibody co-labeled Amberlite microparticles. The trigger and/or activator and/or enhancer can to be adjusted for specific chemiluminescent compounds. For example, 5 uM p-iodophenol can be used in the trigger instead of 8 mM p-hydroxycinnamic acid. If acridinium is used, the activator should be NaOH and peroxide, the concentrations of which are well known to the skilled in the art (e.g. 0.1% H₂O₂, 0.05M NaOH). If 1,2-dioxetane AP substrates are used, the activator can be alkaline phosphatase instead of peroxidase and the enhancer can be TBQ.

In one format, the activator is used as chemiluminescent moiety to couple with affinity groups to form the chemiluminescent binding pair and the chemiluminescent compounds for the activator is added in the assay mix to trigger the CL. For example, if the activator is peroxidase such as horseradish peroxidase, it can be coupled to an antibody or co immobilized on solid phase support with antibody to form the chemiluminescent binding pair. The assay mix can contain luminol, H2O2 and enhancer to trigger the CL. Example of activator are listed above as well as described in many reference such as those described in 20070264664 application. Examples of the chemiluminescent compounds include but not limited to acridan, acridinium, ABEI, luminol, isoluminol and dioxetane type compounds. In principle, any compounds/molecules/particles that can trigger the chemiluminescent can be used as activators. Some activators need to work with certain chemiluminescent compound for light emitting. For example, peroxidase and luminol is this kind of pair. Luciferase-luciferin is also this kind of pair. Therfore luciferase coupled with affinity groups can also be used as chemiluminescent binding pair either by directly coupling or via a linker or on a solid phase support. The reaction mix need to contain the luciferin and other molecules needed for the CL reaction. For example, both ATP and firefly luciferin is needed for the firefly luciferase type chemiluminescent binding pair. As described above, there are several types of luciferase and each of them needs their own type of luciferin. For example, firefly luciferase and click beetles luciferase need firefly luciferin or its analogue as well as ATP and Mg2+ for CL. Bacterial luciferase needs bacterial luciferin or its analogue, which is a reduced riboflavin phosphate (FMNH₂), which is oxidized in association with a long-chain aldehyde, oxygen. Cypridina luciferase needs cypridina luciferin or its analogue. Renilla luciferase use coelenterazine or its analogue. The activator can also be gold nanoparticles since it can trigger the chemiluminescent of luminol, isoluminol and acridan or the like the like at the presence of peroxide compound such as H₂O₂ or other molecules that can emit light at the presence of gold particle and peroxide.

Furthermore, many enzymes/molecules can convert the non-chemiluminescent substrate to chemiluminescent molecules which can emit light spontaneously or emit light under contain condition. These enzymes/molecules can also be used as activators to form chemiluminescent binding pair. The assay mix needs to contain the corresponding non-chemiluminescent substrate. For example, the invention described above illustrated many firefly luciferin derivative/conjugate molecules for certain enzyme detection. These enzymes convert these firefly luciferin derivative/conjugates to free firefly luciferin, which is a chemiluminescent molecule. Therefore these enzymes can be used to form the chemiluminescent binding pair. These enzymes are not limited to luciferin system. Any enzyme that can convert a non-chemiluminescent substrate to chemiluminescent molecules is suitable for the current invention. For example, acridan derivatives and 1,2-dioxetane derivatives can also be used as non-chemiluminescent substrate. Many vendors (e.g. lumigen) provide such acridan derivatives and dioxetane derivatives. For example, alkaline phosphatase can be use to trigger Lumigen APS-5 and Lumigen® PPD, Lumi-Phos® 530, Lumi-Phos® Plus to generate CL. Beta galactosidase can be used for Lumi-Gal 530 substrate. There are also many acridinium derivatives, acridan derivatives, 1,2-dioxetane derivatives, luciferin derivatives or the like can be used as substrate for certain enzyme to produce CL. Many of them are described in patents and literatures as well as the current invention. For example U.S. Pat. No. 6,586,196, US Patent Application 20070048812, 20070015790, 20060257863 and 20060073529 disclosed many chemiluminescent substrate for certain enzymes. These substrates or the like and the corresponding enzyme pair can be used in the current application described above. When 1,2-dioxetane derivatives type substrate is used, dioxetane enhancer such as onium groups (e g ammonium, cationic homopolymer or copolymer comprising positively charged onium groups, (vinylbenzyldimethylbenzylammonium chloride) (BDMQ), poly(vinylbenzyltrimethylammonium chloride) (TMQ), poly(vinylbenzyltributylammonium chloride) (TBQ), poly(vinylbenzyltri(n-pentyl)ammonium chloride) (TPQ), poly(vinylbenzyltributylphosphonium chloride) (TB), poly(vinylbenzyltrioctylphosphonium chloride) (TO) can be coated to the microparticle/nanoparticles of the fluorescent binding pair to enhance the CL of dioxetane.

One of the formats is to use peroxidase as chemiluminescent binding pair and the chemiluminescent molecule that can be triggered by peroxidase (e.g. luminol, isoluminol, acridan and etc. and if necessary, their enhancer) is in the assay buffer. The chemiluminescent molecule can be added at the beginning or only at the detection stage.

Another format is to coat or encapsulate firefly luciferin on or into the solid phase support (e.g. microwell plate or micro particles), and the first antibody is also coated on the solid phase support, the second antibody is conjugated to firefly luciferase. When the analyte is present, the formed sandwich structure will generate luminescence. In another example, the first monoclonal antibody (1^{st} affinity group) for human chronic gonadotropin (hCG) is coated to the micro well plate and firefly luciferin is encapsulated in the plate too, the second monoclonal antibody (2^{nd} affinity group) for hCG, which recognizes a different portion of the hCG molecule than that recognized by the first monoclonal antibody is coupled with firefly luciferase. 200 uL of PBS (pH 8.5) 50 mM; BSA 1 mg/mL, firefly luciferase-antibody conjugate 1 μg/mL is added to microwell, the sample containing hCG is also added and incubated at 25 degree C. for 20 minutes. Next, ATP (1 microliter of 1 mg/mL) is added and the detection window is facing the bottom of the well for the detection.

It is need to know that in the current invention the term coupled with means either directly couple or couple via a linker or co immobilized on the solid support (e. g. nano/micro particle, microwell plate and etc) as described above.

The current invention also discloses a chemiluminescent method for analyte detection. The chemiluminescent dioxetane-generating enzyme is coupled with affinity group (e.g. 1st antibody) to form the chemiluminescent binding pair, dioxetane enhancer moiety and affinity group (e.g. 2^{nd} antibody) is co immobilized on a non-fluorescent solid phase (e.g. non fluorescent micro/nano particle, microwell plate) as enhancing pair. The chemiluminescent binding pair, enhancing pair and analyte can form a sandwich structure complex upon binding. When the dioxetane substrate is added to the assay mix, the complex will emit strong enhanced chemiluminescent light for detection. The coating method of dioxetane enhancer can be readily adapted from the protocol described above by using non-fluorescent particle/surface instead. In one embodiemtn described in example 5, the assay is to detect the peptide human chronic gonadotropin (hCG) in the serum sample.

The current inventions also disclose a homogenous chemiluminescent assay involving the CL enhancer-producing enzyme. One of the binding pair is CL enhancer-producing enzyme-affinity group conjugate and the second binding pair is the peroxidase-affinity group conjugate. This pair can form a sandwich structure of CL enhancer-producing enzyme-affinity group conjugate—analyte—peroxidase-affinity group conjugate at the presence of the analyte. Because the enhancer-producing enzyme and peroxidase are close to each other due to analyte binding, the substrate of the enhancer-producing enzyme (pro enhancer) will become the enhancer molecule, which will then be converted to an active form of enhancer (which can trigger/enhance the CL reaction of a chemiluminescent compound) by the peroxidase. When the chemiluminescent compound is present (e.g. luminol, acridan), it will emit light indicating the presence of the analyte. Suitable enhancer include but not limited to the enhancer for luminol or acridan/peroxidase system, such as those described in U.S. Pat. No. 6,602,679 and U.S. Pat. No. 5,306,621(e.g. p-iodophenol, p-hydroxycinnamic acid or p-imidazol-1-ylphenol, p-phenylphenol). The pro enhancer can be any molecule that can be converted to an enhancer molecule by certain enhancer-producing enzyme. For example, p-iodophenol phosphate for alkaline phosphatase to generate p-iodophenol; p-iodophenol beta-galactoside for beta-galactosidase. Many more can be found in U.S. Pat. No. 5,306,621. The chemiluminescent compound can be any molecule that can emit light by the trigger/enhancement of the enhancer (e.g. luminol, isoluminol, TCPO, acridan used in peroxidase chemiluminescent system). One can also add some CL inhibitor such as scavengers of reactive oxygen and free radicals (e.g. p-nitrophenol or SOD or those described above) to the assay mix to reduce the background caused by the CL from none binding enhancer-producing enzyme and none binding peroxidase. Compound/molecules (e.g. heme, gold nano particle and more described previously) that can function like peroxidase can also be used as peroxidase instead.

In one example the assay is to detect the peptide human chronic gonadotropin (hCG) in the serum sample. All the reagents can also be added together at the beginning. Either set horseradish peroxidase/antibodyl or set alkaline phosphatase/antibody 2 can be immobilized on solid phase. These two set can also be in the form of particle (either one of them or both). Detergents or polymeric surfactants (e.g. Polymeric cationic surfactants including those comprising quaternary ammonium and phosphonium salt groups or onium groups) or CL enhancer (such as ammonium, cationic homopolymer or copolymer comprising positively charged onium groups, BDMQ, TMQ, TBQ, TPQ, TB, Sapphire, Sapphire 11, THQ, phosphonium polymers, and/or copolymers of ammonium and/or phosphonium monomers or the like) can be coupled with either alkaline phosphatase—antibody conjugate or horseradish peroxidase—antibody conjugate or both; or co immobilized with them on solid phase.

The alkaline phosphatase—antibody conjugate or horseradish peroxidase—antibody conjugate or both can also be coupled with a fluorescent moiety which can be excited by luminol chemiluminescent light and therefore the detection will be for the fluorescence light. As described above, using different fluorescent moiety can allow multiplex detection. A luminol CL inhibitor p-nitrophenol or SOD can also be added to the assay mix to reduce the background caused by the non-binding alkaline phosphatase and horseradish peroxidase.

The current inventions also disclose a method used for the homogenous chemiluminescent or fluorescent assay cited or described in the current inventions. In these assays, when the fluorescence or CL for detection is localized in a layer of surface (e.g. the light emitting generated from analyte binding is on the surface the microwell plate); and the detector or light collecting device for the detector (e.g. window, mirror, optic fiber) is placed close to the light emitting layer and far away to most of the background light such as the light generated from none analyte binding (e.g. the detector is placed close to the bottom of the transparent microwell plate instead of on top of the microwell plate); a light absorbing/blocking reagent can be added to the assay mix to reduce these back ground light and therefore increase the relative detection for the analyte. The reason is for example, the fluorescence or CL for detection is generated from the bottom surface of the transparent microwell plate because the affinity group is coated on the bottle of the microwell, when the detector is placed under the well, the light generated from the bottle surface will be absorbed less by the a light absorbing reagent added because the light will encounter less light absorbing reagent since the light source is close to the surface, while the light generated from other part (so it is not caused by analyte binding) such as those from the upper part of the solution in the well will have to travel a much longer distance in the assay mix before it reach the detector therefore it will have a much bigger chance to encounter the a light absorbing molecule and be absorbed.

The principle is to add certain compounds or particles that can absorb the light for detection; preferably they will have high abs for the wavelength of the light for detection. They can be light blocking reagent instead of light absorbing reagent. Examples of them include dye, pigment, and quencher, light absorbing particles, light reflecting particles. It can be done by adding one species of them or by adding the combination of several species (e.g. several dye). Because different dye/pigment/quencher has different absorption coefficient for different wavelength, the concentration (and combination if use multiple dye/pigment/quencher) of them in different application need to be adjusted accordingly to reach the best signal noise ratio. One can start from the concentration that almost completely absorb light generated then decrease the concentration until it does not or only slightly decrease light generated from analyte binding but greatly decrease the light generated not from analyte binding. For example, if the light for detection (not a fluorescence type assay) is from the captured renilla luciferase on microwell surface forming analyte binding sandwich structure, McCormick food coloring red or yellow dye can be added at the concentration of 0.005%˜0.0005%. QSY-35 quencher (abs max 470 nm) can also be used. When acridinium (Em 420 nm) is the direct light source for detection, DABCYL acid quencher can be added since it has high abs at 425 nm. When firefly luciferase is the direct light source for detection, QSY-9 quencher (abs max 562 nm) can be used. If light for detection is generated not from a surface, e.g. the final chemiluminescent or fluorescent sandwich structure is not captured on a layer of surface (e.g. they are evenly distributed in the assay solution), this method will not work. Particles can absorb or block the light can also be used. For example, magnetic micro particles containing high content of Fe oxide can also be added, because they have dark color and are not transparent, they can absorb and block the light nearby since they can suspend in the solution. Another choice is fine carbon powder; they can also absorb light. Furthermore they can absorb the molecules that can generate CL or fluorescence but not bind with analyte therefore reduce their light generating capability. Other absorbent that can absorb the molecules that generating back ground light can also be added. For example, amine coated beads can bind with firefly luciferin, therefore if the assay use firefly luciferase-affinity group—analyte—luciferin generating enzyme-affinity group, this beads can be added to reduce the back ground.

In one example, the assay is performed based on the Example 12 of 20070264664 application, the modification is that the luminomter detector window is placed right underneath the well to detect the bottom of the well only and McCormick food coloring red dye is added to reach the concentration of 0.01%.

Using dark magnetic micro particles can also help the assay speed by providing string/mixing effect to the assay solution if a rotating/moving magnetic field is applied to the assay solution because the magnetic beads will keep on moving to function as an internal shaker/mixer. If the detection is focused on the bottom surface of the well, a magnetic field can be applied to guide the magnetic micro particles to form a layer to cover the bottom surface therefore block the light form the solution above, which is the background light. Dye/pigment can also be coated on or trapped in the micro particles to enhance their light absorbing/blocking capability. None magnetic light absorbing/blocking particles can also be used by mechanical means such as using gravity or centrifuge to form a light-blocking layer to function as the same.

The analyte binding and therefore the light emitting can also be placed on magnetic microparticle instead on the surface of the microwell. In the detection step, a magnetic field is need to be applied to move the magnetic particle close to the detector window. In one example, the assay is performed based on the Example 14 of 20070264664 application, the modification is that the luminomter detector window is placed right underneath the well and a magnetic field is applied to enable the magnetic particle to form a layer at the bottom of the well before the detection. It is preferred that the formed magnetic particle layer covers all the bottom of the well or cover all the area of the window of the detector. Here the dark color magnetic particle itself function as a light blocking for the light from upper layer solution. McCormick food coloring red dye can also be added at the concentration of 0.005% to further reduce the background. Other light absorbing/blocking beads can also be used as solid support as long as they can form a light-blocking layer by certain means. Other nano/microparticle can also be used as solid support as long as they can form a light-generating layer by certain means (e.g. centrifuge); if the particles can absorb/block light, a dye may not be needed to add to the assay mix; if the particle is transparent, the light absorbing/blocking reagent will be needed in the assay solution.

### EXAMPLES

### Example 1

The first monoclonal antibody (1^{st} affinity group) for human chronic gonadotropin (hCG) is coated on 1 um size carboxylate polystyrene micro particle which is then coated with dioxetane enhancer, the second monoclonal antibody (2^{nd} affinity group) for hCG, which recognizes a different portion of the hCG molecule than that recognized by the first monoclonal antibody is coupled with alkaline phosphatase.

The detection solution contains the following:

| | | |
|---|---|---|
| PBS buffer (pH 8.5) | 50 | mM |
| BSA | 1 | mg/mL |
| alkaline phosphatase -antibody conjugate | 2 | μg/mL |
| enhancer particle -antibody conjugate | 6 | μg/mL |

100 microliters of assay solution described above is mixed with 100 microliters of HCG containing sample and incubated at 25 degree C. for 20 minutes. Next, 100 microliters of 1,2-dioxetane AP substrate in 0.01M PBS buffer, 5 microgram/mL is added to the reaction mix and placed in a luminomter for reading the light signal. This assay can be used to detect HCG in a sample.

### Example 2

In another example, the first monoclonal antibody (1^{st} affinity group) for human chronic gonadotropin (hCG) is coupled with horseradish peroxidase, the second monoclonal antibody (2^{nd} affinity group) for hCG, which recognizes a different portion of the hCG molecule than that recognized by the first monoclonal antibody is coupled with alkaline phosphatase.

The assay solution contains the following:

| | | |
|---|---|---|
| PBS (pH 8.5) | 50 | mM |
| BSA | 1 | mg/mL |
| alkaline phosphatase -antibody conjugate | 2 | μg/mL |
| horseradish peroxidase -antibody conjugate | 2 | μg/mL |

100 microliters of assay solution described above is mixed with 100 microliters of HCG containing sample and incubated at 25 degree C. for 20 minutes. Next, 100 microliters of 0.1M sodium borate solution (pH 9.0) containing of 1.0×10⁻⁴ M luminol, 5.00×10⁻⁴ M H2O2 and 5.00×10⁻⁴ M p-iodophenol phosphate is added to the reaction mix and placed in a luminomter for reading. This assay can be used to detect HCG in a sample.

In another example, microwell plate described above is incubated for 30 minutes at 37° C. and washed 3 times. After addition of peroxidase-conjugated antihuman IgG to each well, the plate is incubated again for 30 minutes at 37° C. and without further washing, 10 uL McCormick (aka Schilling) food coloring red dye is added to reach the concentration of 0.01% and the detector is placed under the well. The later steps are the same of those in the reference. Alternatively, peroxidase-conjugated antihuman IgG can also be added at the beginning and no washing step is performed before the CL reaction. Therefore the assay becomes a true homogenous (none washing) multiplex assay.

### Example 3

The first monoclonal antibody (1^{st} affinity group) for human chronic gonadotropin (hCG) is coupled with gold nano particles (50 nm), the second monoclonal antibody (2^{nd} affinity group) for hCG, which recognizes a different portion of the hCG molecule than that recognized by the first monoclonal antibody is also coupled with gold nano particles (5 or 10 nm). These two gold nano particles are mixed together at equal ratio. The assay solution contains the following:

| | | |
|---|---|---|
| PBS (pH 8.5) | 50 | mM |
| BSA | 1 | mg/mL |
| Gold nano particle -antibody conjugate | 5 | μg/mL |

100 microliters of assay solution described above is mixed with 100 microliters of HCG containing sample and incubated at 25 degree C. for 20 minutes. Next, 100 microliters of 0.1M sodium borate solution (pH 10.0) containing of 5.0×10⁻⁴ M luminol, 5.00×10⁻⁴ M H2O2 is added to the reaction mix and placed in a luminomter for reading. A optionally 200 ul 1M NaCl can also be added and incubated for 5 min before adding luminol and H2O2. 2×10⁻⁵M hydrazine can also be used instead of H2O2 to initiate luminol chemiluminescence. Once can establish a CL intensity vs HCG concentration curve by testing samples containing series amount of HCG and use this curve to determine the HCG amount in a unknown sample. Alternatively, other metal particle that can catalyze the CL reaction such as platinum colloids can also be used instead of the gold nano particle.

### Example 4

The first monoclonal antibody (1^{st} affinity group) for human chronic gonadotropin (hCG) is coupled with QD 650, the second monoclonal antibody (2^{nd} affinity group) for hCG, which recognizes a different portion of the hCG molecule than that recognized by the first monoclonal antibody is also coupled with alkaline phosphatase. The alkaline phosphatase-antibody conjugate is immobilized on polystyrene micro well surface. 200 uL of PBS (pH 8.5) 50 mM; BSA 1 mg/mL, QD 650-antibody conjugate 6 μg/mL is added to microwell, the sample containing hCG is also added and incubated at 25 degree C. for 20 minutes. Next, 50 uL of 1,2-dioxetane AP substrate in 0.01M PBS buffer, 5 ug/mL containing Sapphire enhancer and McCormick blue food coloring dye (or QSY-21 dye) 5 ug/mL is added to the reaction mix. The detection window is facing the bottom of the well to collect the light signal at >600 nm.

### Example 5

The first monoclonal antibody (1^{st} affinity group) for human chronic gonadotropin (hCG) is coated to the micro well plate, the second monoclonal antibody (2^{nd} affinity group) for hCG, which recognizes a different portion of the hCG molecule than that recognized by the first monoclonal antibody is coupled with renilla luciferase. 200 uL of PBS (pH 8.5) 50 mM; BSA 1 mg/mL, renilla luciferase-antibody conjugate 1 μg/mL is added to microwell, the sample containing hCG is also added and incubated at 25 degree C. for 20 minutes. Next, coelenterazine (1 of 1 mg/mL) is added and the detection window is facing the bottom of the well for the detection. McCormick food coloring red or yellow dye will be added to reach the final concentration of 0.005%˜0.0005%. QSY-35 quencher (abs max 470 nm) can also be used.

What is claimed is:

1. A method to detecting analyte in a sample, comprising:
a) contacting said sample with a first ligand of the analyte coupled with luminescence producing enzyme and a second ligand of the analyte coupled with solid phase support encapsulated with chemiluminescent dye, and
b) detecting the light generated from said luminescence.
